(19) United States
(12) Patent Application Publication
(10) Pub. No.: US 2006/0173268 A1
(43) Pub. Date: Aug. 3, 2006

(54) Methods and systems for controlling acquisition of images

(75) Inventors: Rakesh Mullick, Bangalore (IN); Christopher Judson Hardy, Niskayuna, NY (US); Robert David Darrow, Scotia, NY (US); Raghu Kokku, Secunderabad (IN); Timothy Poston, Bangalore (IN)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/045,838

(22) Filed: Jan. 28, 2005

(51) Int. Cl. A61B 5/05

(52) U.S. Cl. 600/407

(57) ABSTRACT

Systems and methods for interacting effectively with three-dimensional data are provided such that a data acquisition system of an imaging system can be guided appropriately to gather relevant information from the object being imaged. In one embodiment, the imaging system includes the data acquisition system for obtaining a three-dimensional image of the object; and a processor coupled to the data acquisition system. The processor may be configured for receiving a user interface input based on interaction with the three-dimensional image, and for providing multiple parameters to the data acquisition system based on the user interface input. These parameters may be used for further acquisition by the data acquisition system.

### BACKGROUND

The invention relates generally to the field of imaging and more specifically to the methods and systems for incorporating a user interface input based on a three-dimensional image for directing data acquisition in an imaging modality in order to acquire data descriptive of a three-dimensional structure being imaged.

There are several imaging modalities for imaging an object and obtaining relevant information related to internal features of the object. These include X-ray imaging, computed tomography (CT), magnetic resonance imaging (MRI), magnetic resonance spectroscopy, and ultrasound imaging. Though these imaging modalities acquire three-dimensional data about the object, the user interaction with the image is limited to the two dimensional space of the monitor surface (for output) and mousepad (for input). Due to this limitation, as explained below in more detail, several such sessions may be needed to acquire the necessary information from the object.

An important aspect of an imaging process in any of the above imaging modalities is the choice of which data to acquire, with reference to precisely which sets of points in space. This typically involves a cycle of interaction between what the user wishes to know; what settings are given to the imaging system, and then fixing mechanical positions, field strengths, pulses and frequencies, and the like, and deciding the image acquisition process. Any scan thus typically begins with the collection of a ‘scout’ image, consisting of one slice, several slices, or enough parallel slices or two dimensional phase encodes to constitute ‘volume data’, chosen in a region within which the target structure is known to lie, though exact positioning is not yet available. The resulting display of planar images is used for selecting further features. However, there are several constraints in the current process for determining further data acquisition. Most of these arise because of the fact that the user has to use planar images and interact with the images using a two-dimensional mouse interface. Thus user has typically just two degrees of freedom with which to operate and select the desired features. Two degrees of freedom adjustable by the side-to-side and front-to-back motion of a mouse cannot simultaneously control the larger set of parameters needed to specify a data collection geometry in three dimensions. In the current user interaction techniques, the user must repeatedly switch (by clicks, by motion to a different sub-window, and the like), between signaling motion in different two-dimensional combinations of the six position quantities that can change independently, i.e. x, y and z directions, roll, pitch and yaw. This limitation leads to a time-consuming iteration process. Time spent with costly equipment is costly, and in medical instances is stressful for the patient. These are some exemplary constraints of the current interaction with three-dimensional data.

Therefore there is a need for a technique where a user can interact more effectively with the three-dimensional data in an imaging process and direct the data acquisition system accordingly for acquiring relevant images.

### BRIEF DESCRIPTION

Briefly, in accordance with one aspect of the present technique, an imaging system includes a data acquisition system for obtaining a three-dimensional image of an object, and a processor coupled to the data acquisition system. The processor may be configured for receiving a user interface input based on interaction with the three-dimensional image, and for providing multiple parameters to the data acquisition system based on the user interface input. These parameters may be used for further acquisition by the data acquisition system.

According to another aspect, a method of acquiring three-dimensional data in an imaging modality is provided. The method includes steps of obtaining a three-dimensional image of an object being imaged; receiving a user interface input based on interaction with the three-dimensional image; and providing multiple parameters to a data acquisition system based on the user interface input.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatic representation of an exemplary embodiment including a virtual user interface and an imaging system;

FIG. 2 is a diagrammatic representation of an exemplary Magnetic Resonance Imaging (MRI) system used in one exemplary embodiment of the present technique;

FIG. 3 is a diagrammatic representation of three planes of an object used for image acquisition;

FIG. 4 is a diagrammatic representation of a first planar view of an image from image acquisition of FIG. 3;

FIG. 5 is a diagrammatical representation of a second planar view of an image from image acquisition of FIG. 3;

FIG. 6 is a diagrammatic representation of a third planar view of an image from image acquisition of FIG. 3;

FIG. 7 is a diagrammatic representation of defining an oblique plane in the image of FIG. 4;

FIG. 8 is a diagrammatic representation of defining a oblique plane in the representation shown in FIG. 7;

FIG. 9 is a diagrammatic representation of defining a doubly oblique plane in the image of FIG. 6;

FIG. 10 is a diagrammatic representation of defining a double oblique plane in the representation shown in FIG. 9;

FIG. 11 is a diagrammatic representation of a scout image in multiple planar slices;

FIG. 12 is a diagrammatic representation of an exemplary user menu showing exemplary tools to specify the next acquisition protocol; and

FIG. 13 is a diagrammatic representation of an exemplary view obtained by slicing the planar views of FIG. 11.

### DETAILED DESCRIPTION

Aspects of the present technique include an application of a hand-immersed virtual reality or a reach-in environment for real-time management of three-dimensional data acquisition using one or more imaging modalities.

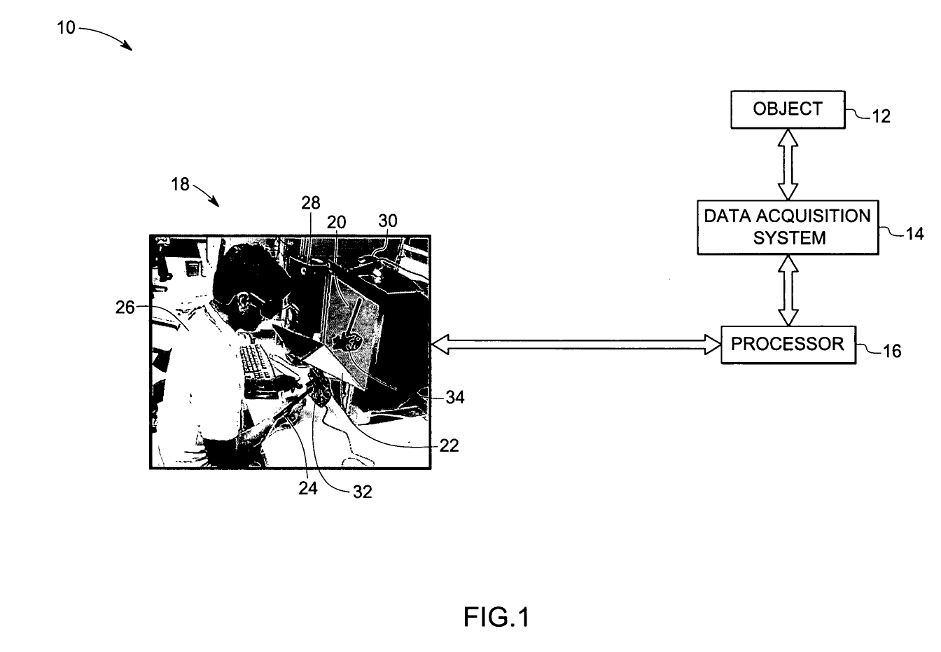

FIG. 1 is a diagrammatic representation of an exemplary imaging system 10 employed for imaging an object 12 via a data acquisition system 14. The object 12 may be a patient, an industrial part, a geographical region, an underground rock, a pipeline, an item of baggage, a biological sample or any other three-dimensional structure. The data acquisition system 14 is used in particular for obtaining a three dimensional image of the object 12. A processor 16 is coupled to the data acquisition system 14 and to a virtual user interface 18 according to aspects of present technique. The processor 16 is configured for receiving the three-dimensional image from the data acquisition system and for providing one or more scanning parameters to the data acquisition system 14, based on user interface input received via the virtual user interface 18. These parameters are used for further acquisition by the data acquisition system, according to aspects of the present technique. The virtual user interface includes a computer workstation 20 configured for displaying a three-dimensional image 34 of the object 12. The three dimensional tracking device 24 may be coupled to the computer workstation 20 and configured for allowing up to six degrees of freedom (DOF) of movement to a user 26, and communicating such movement to the computer workstation 20. In one example, the three dimensional tracking device 24 is configured with at least one button which the user may click or hold down to signal choice of an entity, dragging, and the like. Optionally the three-dimensional tracking device 24 may also be configured with one or more devices capable of a scalar output, such as a sensor that reports the force of pressure (not merely the Yes/No of clicking), or a slider, that the user may use to communicate a graded rather than discrete intention. The virtual user interface also includes a virtual display set-up, shown as workspace 32 in FIG. 1 coupled to the computer workstation 20 and configured for allowing the user to reach-in and interact with the three-dimensional image of the object via the three-dimensional tracking device 32. As used herein, the term ‘reach-in environment’ refers to a virtual reality, or stereo display in which the user perceives positions and motions of an element in the display as being the positions and motions of the hand-held sensor, rather than translated or rotated versions of the said positions and motions. The term does not require, though it does not exclude, the property of ‘head immersion’ by which the display space is perceived as fully surrounding the user. Such head immersion is often taken to be a required aspect of ‘virtual reality’, but in an exemplary embodiment, the head-immersion may be omitted, thereby avoiding various problems of simulator sickness, isolation from other workers, and so on.

Stereo display as herein referred to is an established technology well known to those skilled in the art, which presents a different image to each eye, with the differences corresponding to those that result from the eyes' different location. These images in one example, may be photographed images using cameras located at the intended eye position, or as in another example, the images may be generated by computer from scan data. The human visual perception in all these scenarios generates a sense of the depth (distance from the eye) of each point on each object in the scene. Using a filter mounted in front of each eye, according to aspects of the present techniques, a stereo view may be displayed on a large screen (as in 3D-IMAX), or on a computer display. Sufficiently small displays may be advantageously mounted separately in front of each eye, removing the need for filters.

In operation, a user 26 views in stereo, for example via 3D glasses 28 or other known virtual visualization devices, the data 34 acquired to date. Optionally the data may be a scout image or a model image representing the object 12 and displayed on the computer workstation 20 and the workspace 32. The data may include for example, a generic patient geometry, optionally adapted to demographic data concerning gender, age, height and weight. In one example, using a medical imaging modality, such generic data may be displayed in the workspace 32 before any scan begins for a patient. Similarly, if an industrial part needs to be scanned, and a computer aided design (CAD) file of the part is available, the part may be installed in a standardized holder and the geometrical details of the ‘ideal’ part from the CAD file may be displayed in the workspace 32 before scanning of the actual part begins, for analysis, search for defects, or other reasons. The scanning modalities for industrial applications may be computed tomography (CT) for study of X-ray absorbency, a magnetic resonance elastography of mechanical properties, or another scanning modality known to those skilled in the art. Similarly, again, if the data are seismographic, a three-dimensional model of the topography of the area from surface, airborne or satellite surveying may be used and displayed at the workspace 32.

As mentioned above, the data may be viewed in stereo in the workspace 32, which may be described as a stereoscopic three-dimensional workspace, where hand actions may grasp geometric structures such as planes and rectangular boxes that appear in positions matching the hand's, and move accordingly. In one example, the workspace 32 is a reflected region in a sloping mirror 22 so that the user's hands can move a tracking device 24 in the workspace 32 without striking or masking the display 34, viewed in the mirror 22 through shutter glasses 28 synchronized with the workspace 32 via an electromagnetic emitter 30. Optionally, there may be a second tracking device, for use with the other hand. All devices may be connected to a shared processor 16. Tracking device may be a stylus, a mechanical robot arm, electromagnetic sensing device using an optical camera image, or such other three-dimensional tracking devices as are known to one skilled in the art. Each tracking device 24 may have at least one button whose state (pressed or unpressed) and its changes are reported to the processor 16 and is used to determine the interactions between the device 24 and the structure being selected for image acquisition by the user. For example, if the structure is a rectangle defining an option for a planar set of points at which new data may be acquired, holding down the button may signal ‘drag the displayed rectangle with my hand’ by locking the geometrical relation between the displayed rectangle with the sensor, while a button click may signal ‘acquire the data corresponding to the present position’.

In embodiments of the user interface 18, the user may grasp the displayed geometry to displace or rotate it, and zoom it by the use of a scale slider or by grasping a point on it by using the three-dimensional tracking device in each hand. With these controls the user may quickly bring a desired region into view at a convenient scale. The current position and geometry of the selected structures or the features of the three-dimensional image, according to aspects of the present technique, controlled through natural human hand-eye coordination, are thus transformed by the processor 16 into spatial specifications, i.e., one or more parameters to be used by the data acquisition system 14 for further image acquisition. The parameters may include for example specifications for gradient fields and pulse sequences in magnetic resonance imaging (MR), phase pattern in a system directed by phased array emission such as certain ultrasound and radar systems, or repositioning of mechanical components. The acquired data may again be presented in the form of a new three-dimensional image scene on the monitor 20. The user may again select further details of the image for more information. The user is also provided with an ability to alter the apparent viewpoint by virtual grasping and moving the scene as a whole, or translating and/or rotating the collective position in which the geometric elements of the data display appear.

The virtual user interface environment as explained herein, may include optionally different controls for immediate image analysis processes such as segmentation (identifying particular three-dimensional regions as components of vasculature, probable tumor, shale, and the like), and other geometrical/topological queries such as connectivity, for example, clicking on two points and inquiring whether they can be connected via a path that remains within the segment (anatomical connectivity applications). Where the segment has directional aspects that the system can attribute correctly, such as classifying a nerve as efferent or afferent or determining the direction of flow in a blood vessel or aquifer, the analysis result for such a point pair may include information as to whether one point is ‘downstream’ of the other (directed connectivity applications). The interface may also include tools by which to modify the results (such as bridging a gap between two artery segments that can only be due to bad or incomplete data), or to use the results to guide further acquisition of a region or slice selected automatically or by the user to contain or pass through the component found.

In applications where multiple simultaneous scanning modalities may be used, the interface may advantageously include user tools for invoking mutual registration of the images differently acquired, for correcting such registration on, for example, anatomical grounds apparent to the user, and for controlling the next acquisition in one modality by reference to a segment identified in another modality.

The imaging system 10 as described herein may be an MRI system, an MR spectroscopy, a CT system, an ultrasound system, an X-ray imaging system, a radar system, a seismological system, an optical system, a microscope, a positron emission detection system, any other three-dimensional image acquisition system that is now or may become available, or a combination thereof.

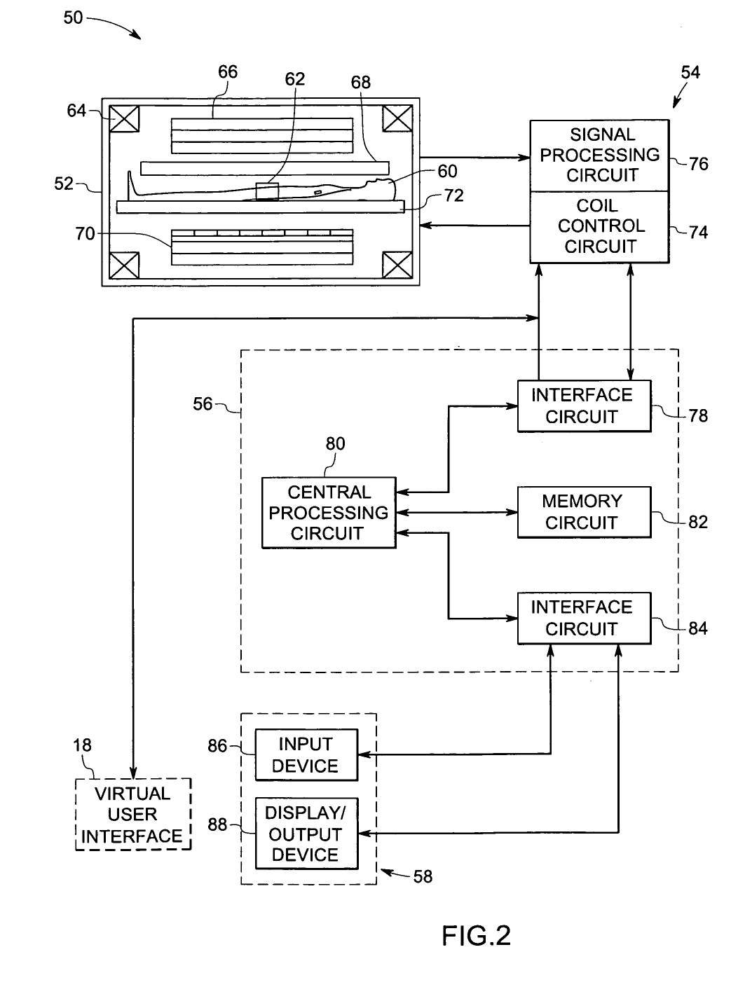

FIG. 2 is a diagrammatic representation of an exemplary MRI system used in one exemplary embodiment of the present technique. The magnetic resonance system, designated generally by the reference numeral 50, is illustrated as including a magnet assembly 52, a control and acquisition circuit 54, a system controller circuit 56, and an operator interface station 58. The magnet assembly 52, in turn, includes coil assemblies for selectively generating controlled magnetic fields used to excite gyromagnetic materials spin systems in a subject 60 or more specifically in the region of interest 62. In particular, the magnet assembly 52 includes a primary coil 64, which typically includes a superconducting magnet coupled to a cryogenic refrigeration system (not shown). The primary coil 64 generates a highly uniform B0 magnetic field along a longitudinal axis of the magnet assembly. A gradient coil assembly 66 consisting of a series of gradient coils is also provided for generating controllable gradient magnetic fields having desired orientations with respect to the anatomy or region of interest 62. In particular, as will be appreciated by those skilled in the art, the gradient coil assembly produces fields in response to pulsed signals for selecting an image slice, orienting the image slice, and encoding excited gyromagnetic material spin systems within the slice to produce the desired image. In MR spectroscopy systems these gradient fields may be used differently. An RF transmit coil 68 is provided for generating excitation signals that result in MR emissions from the subject 60 that are influenced by the gradient fields, and collected for analysis by the RF receive coils 70 as described below.

A table 72 is positioned within the magnet assembly 52 to support a subject 60. While a full-body MRI system is illustrated in the exemplary embodiment of FIG. 2, the technique described below may be equally well applied to various alternative configurations of systems and scanners, including smaller scanners and probes used in MR applications.

In the embodiment illustrated in FIG. 2, the control and acquisition circuit 54 includes a coil control circuit 74 and a signal processing circuit 76. The coil control circuit 74 receives pulse sequence descriptions from the system controller 56, notably through an interface circuit 78 included in the system controller 56. As will be appreciated by those skilled in the art, such pulse sequence descriptions generally include digitized data defining pulses for exciting the coils of the gradient coil assembly 64 during excitation and data acquisition phases of operation. Fields generated by the transmit coil assembly 67 excite the spin system within the subject 60 to cause emissions from the anatomy of interest 62. Such emissions are detected by RF receive coils 70 and are filtered, amplified, and transmitted to a signal processing circuit 76. The signal processing circuit 76 may perform preliminary processing of the detected signals, such as amplification of the signals. Following such processing, the amplified signals are transmitted to the interface circuit 78 for further processing.

In addition to the interface circuit 78, the system controller 56 includes a central processing circuit 80, a memory circuit 82, and an interface circuit 84 for communicating with the operator interface station 58. In general, the central processing circuit 80 (which typically includes a digital signal processor, a CPU or the like, as well as associated signal processing circuit) commands excitation and data acquisition pulse sequences for the magnet assembly 52 and the control and acquisition circuit 54 through the intermediary of the interface circuit 78. The central processing circuit 80 also processes image data received via the interface circuit 78, to perform 2D Fourier transforms to convert the acquired data from the time domain to the frequency domain, and to reconstruct the data into a meaningful image. The memory circuit 82 serves to save such data, as well as pulse sequence descriptions, configuration parameters, and so forth. The interface circuit 84 permits the system controller 56 to receive and transmit configuration parameters, image protocol and command instructions, and so forth.

The operator interface station 58 includes one or more input devices 86, along with one or more display or output devices 88. In a typical application, the input device 86 will include a conventional operator keyboard, or other operator input devices for selecting image types, image slice orientations, configuration parameters, and so forth. The display/output device 88 will typically include a computer monitor for displaying the operator selections, as well as for viewing scanned and reconstructed images. Such devices may also include printers or other peripherals for reproducing hard copies of the reconstructed images.

A virtual user interface 18 as described in reference to FIG. 1, may be incorporated within the operator interface station 58 or may be used as a separate unit coupled with the operator interface station 58 and with the system controller 76. As explained with reference to FIG. 1, the user may select features or a region of interest from a rapidly acquired volume data in an arbitrary region, e.g., a rectangular region; such data may be used as a scout image. If a scout image is not acquired automatically, the patient position data and scan protocols may be used by the user in the virtual user environment. When an acceptable region has been defined in this way, the user may invoke the acquisition function (by clicking a button, by issuing a voice command, or by such other method as may be familiar to one skilled in the art), and volume data are acquired for spatial points corresponding to the selected region by the data acquisition system, which is the control and acquisition circuit 54 in this embodiment. For the scout stage it may be appropriate to collect data at a relatively coarse resolution, with default frequency settings and visual display parameters chosen to make gross structural features such as bones or underground channels conspicuous and thus helpful in further navigation. However, such settings may also be user-adjustable at this stage. At each change of position by the user, the x, y and z coordinates and the center point of a location selected by the user via the three-dimensional tracking device are transmitted to the data acquisition system. This in turn leads to necessary instructions for the imaging protocol to acquire data from the corresponding plane in the patient being scanned. The resultant images are reported back to the interface system, which displays the data (with suitable assignments of color, transparency, and the like, as will be evident to one skilled in the art). The processor of the virtual user interface may also be configured for storing successive three-dimensional images of different regions of interest obtained during the imaging process. In one example, a movie may be created using these different images to view continually successive three-dimensional images of regions of interest.

Medical applications using aspects of the present technique may include, for example, cardiac imaging applications, surgical applications, internal organ segmentation applications, confocal microscopy for bioscience applications or other similar imaging applications known to one skilled in the art. The imaging system may also be configured to operate with an interventional device to help the user navigate through the patient anatomy during surgery or for targeted delivery of pharmaceuticals.

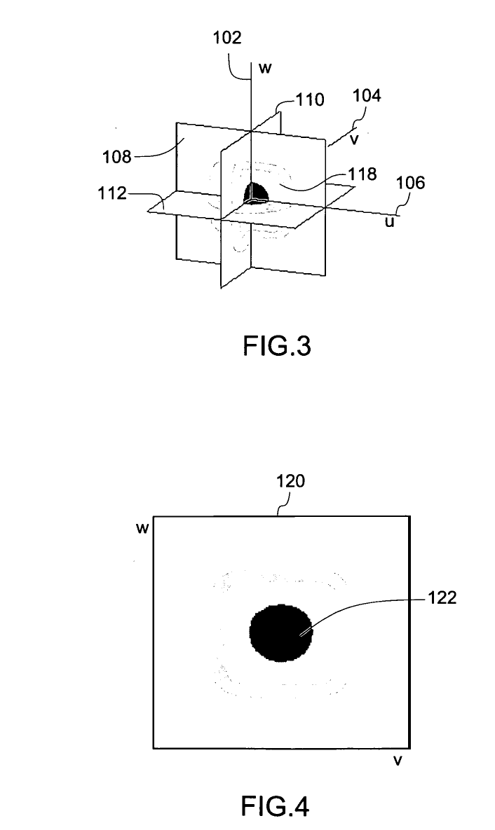

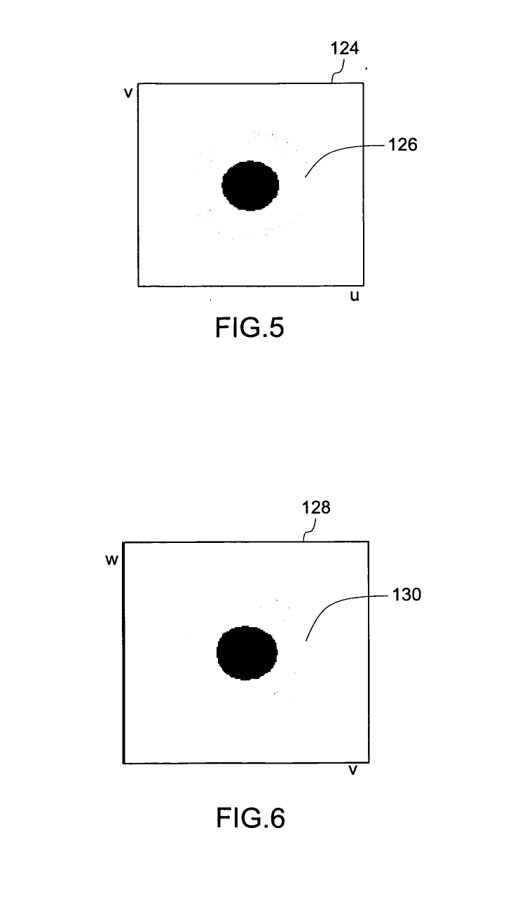

FIG. 3-FIG. 6 show the complexity in visualizing a three-dimensional image based on the two dimensional results obtained currently by using any imaging modality. FIG. 3 is a diagrammatic representation of three planes 108, 110 and 112 in the u, v, and w directions designated by reference numerals 106, 104 and 102 of an object 118 whose image is acquired. The image designated generally by the reference numeral 100 represents three slices in orthogonal directions selected for a scout image by a user. Typically these would be displayed as three different flat (two-dimensional) images as shown in FIG. 4-6. FIG. 4 is a diagrammatic representation of an image 120 showing one view of the object 118 of FIG. 3, designated generally by reference numeral 122. FIG. 5 is also a diagrammatic representation of an image 124 showing another view of the object 118 of FIG. 3, designated generally by reference numeral 126 and FIG. 6 is another diagrammatic view of an image 128 showing another view of the object 118 of FIG. 3, designated generally by reference numeral 130. As can be appreciated from FIG. 3, it is not cognitively simple to mentally place the images 120, 124 and 128 in the configuration 100, and imagine the plane that will meet chosen features of the object 118 in a desired way.

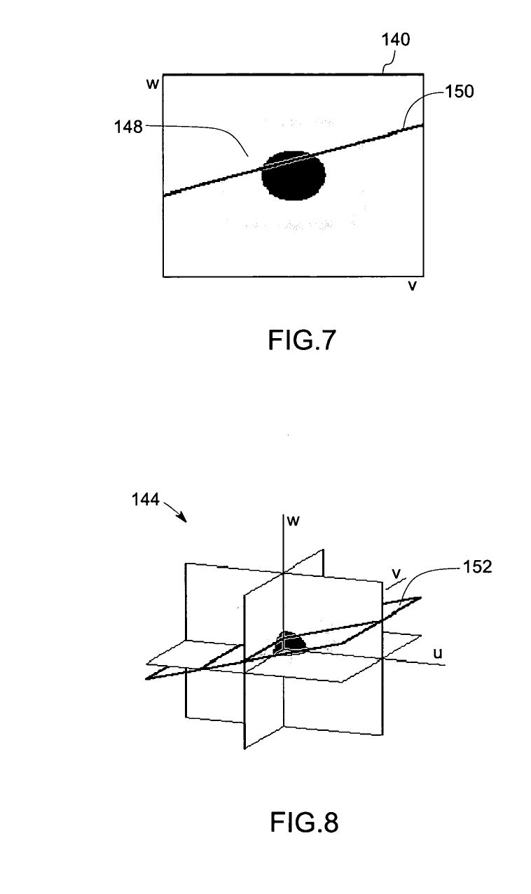

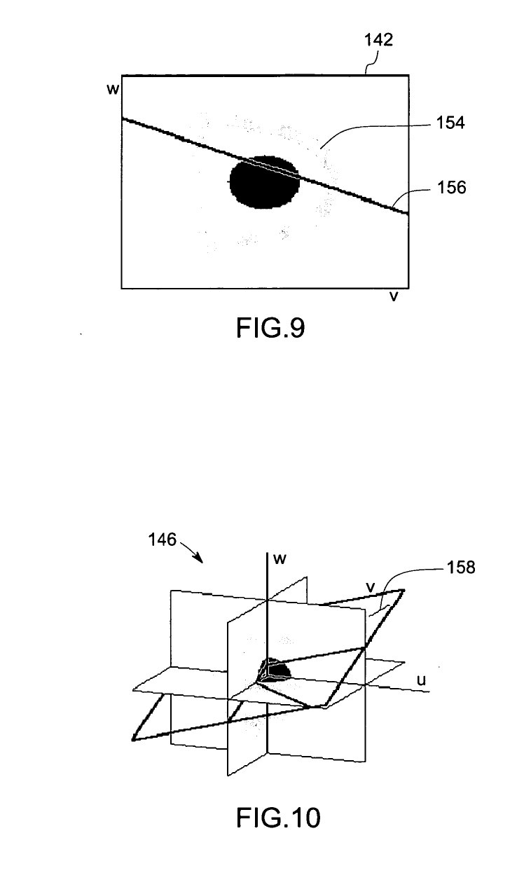

The image reconstruction becomes further complicated if the user wants to select an oblique plane as shown in FIG. 7-10. Thus if from the view 140 as shown in FIG. 7, the user wants to selects an oblique plane as shown by reference numeral 152 in the view 144 in FIG. 8, a typical two-dimensional interface requires the user to select in FIG. 7 a line 150 going through the image 148 as shown in a two-dimensional view 140 in the (u, w) plane. It is a further non-trivial task to imagine in which plane the line 150 must meet the (u, w) plane to produce a desired ‘oblique plane’ 152 in which data will be useful. This become even more complex if the user must select in the view 142 another line 154 in which a desired plane should meet the (v, w) plane, in order to fix a ‘doubly oblique’ plane 158 showing the image 156 as shown in FIG. 9, to ensure selection of a doubly oblique plane 158 as shown in the view 146 in FIG. 10. Commonly, multiple adjustments of the lines 150 and 154 are typically required before the desired image is reached. Thus aspects of the present technique resolve the issues as presented in FIG. 3-FIG. 10 by providing a flexible user interaction technique which provides the user with six simultaneous degrees of freedom in selecting desired features or sections from any image.

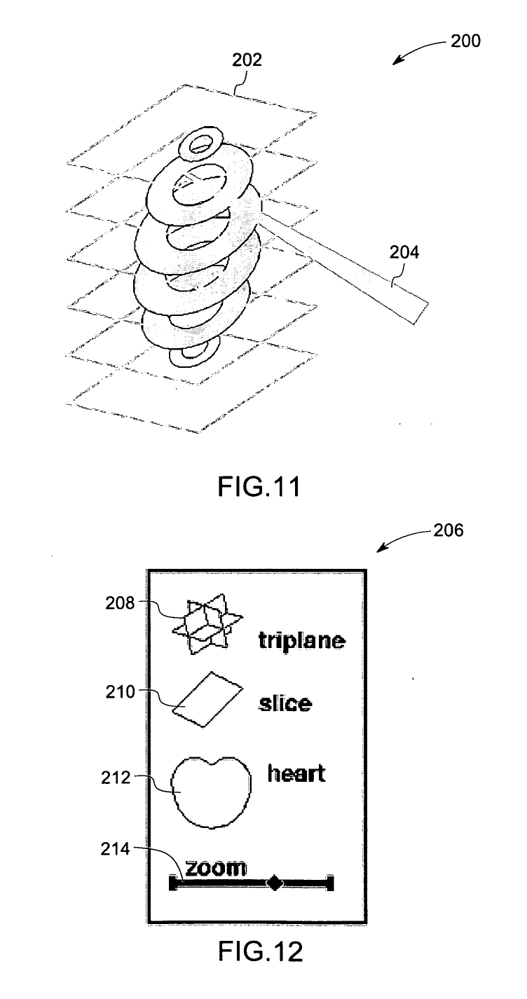

FIG. 11 is a diagrammatic representation of a scout image 200 in multiple planar slices 202 displayed according to aspects of the present technique. As will be evident to one skilled in the art, the scout image may be a single slice, a stack of a moderate number of parallel slices, a group of three orthogonal slices, or another such configuration. A stylus 204 may be used to grasp and move the stack of planar slices 202 for a better view. Optionally a second stylus may be used, controlled by the other hand. The stylus 204 can also be used to select one of the menu items as shown in FIG. 12.

FIG. 12 is a diagrammatic representation of an exemplary user menu 206 showing exemplary tools (user options) to specify the next acquisition protocol by the data acquisition system according to aspects of present technique. The tools shown include, but are not limited to a triplane 208, a slice, 210, a heart 212 or a zoom slider 214. Each tool may generate a specific image selection action associated with it. For example, selecting the zoom slider 214 may allow the user to drag the control to the left, shrinking the display of stack 202, or to the right, enlarging it. In a specific example of cardiac scanning such as by MRI, selecting the heart icon 212 may display a generic heart model, which may be superposed to get a visual fit to the visible slices such as 202 (here stylized as slices of a thick-walled ellipsoid) of the target structure, providing hints to the location of features which are not obviously visible in the slices. This model may be displaced, rotated, and rescaled by interactions similar to those above. In an industrial application of the present invention the heart model could be replaced by a CAD model of the object being scanned. The appropriate modification for other fields of application will be evident to those skilled in the art. Similarly, selecting the triplane icon 210 produces a set of three orthogonal planes, rigidly coupled, which otherwise behave in the same manner as a single plane. The triplane structure may be grasped, moved, and the display on it updated according to the conventions described above for single planes. In addition, by placing the stylus tip close to the intersection point of the three planes the said intersection point can be dragged around, keeping fixed the orientation of each plane the same but moving it to pass through the dragged point. Allowing more than one triplane to coexist in the display is probably unhelpful to the user, and is thus excluded from our preferred implementation.

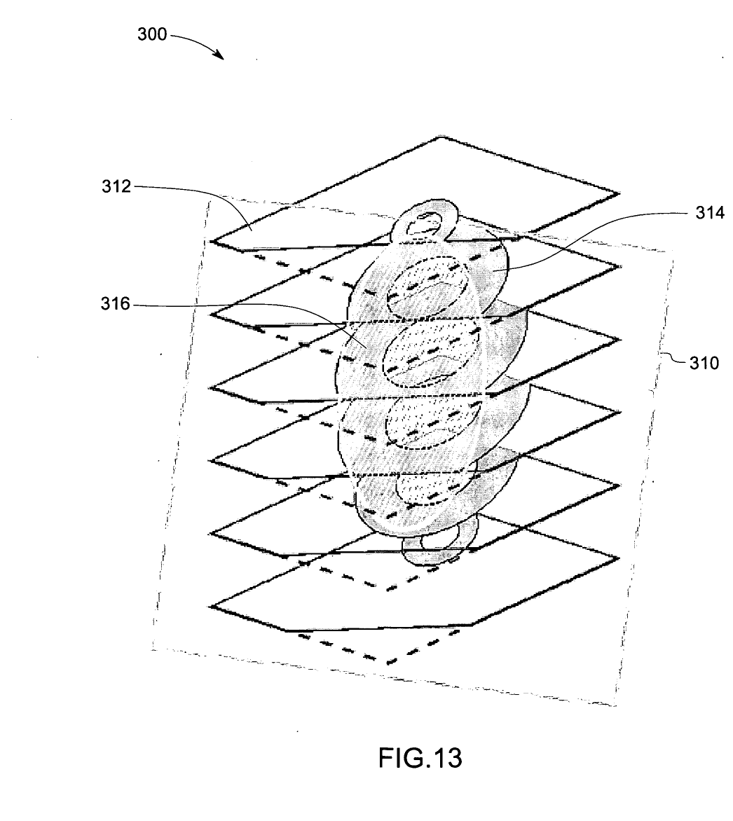

In another exemplary embodiment, selecting the slice icon 210 may produce a plane 310 as shown in FIG. 13. FIG. 13 is a diagrammatic representation of an exemplary view 300 obtained by slicing the planar views 312. In the case of the thick-walled ellipsoid structure 314 used here for illustration, the result is an elliptical annulus or filled ellipse 316, with or without a central hole according to position. If the structure being scanned is in motion, like the heart, the updates may be acquired, transmitted and displayed on the plane 310 at the maximum practical speed, up to the rate of 60 frames per second supported by most display devices. If the structure scanned is moving rhythmically, like the heart, the collection or its display may optionally be ‘gated’ by movement data such as EKG signals, so that the image is always collected at the same phase of motion. The user may leave the plane 310 in place, and select the icon again, producing a second plane 310, which coexists with it in the display, so that multiple selected slices of the scanned structure can be seen simultaneously, with options to remove parts of one slice which are obscuring the user's view of another slice.

An additional menu of buttons, voice commands or similar widgets (not shown) may be used to allow ‘instant replays’ in real time or slowed motion of changing data just recorded. All of these views change, in terms of what appears on the screen though not in terms of the data represented, when the whole assembly is rotated or displaced within the work volume (not changing the relation of each individual part to the data acquisition system). The user thus has movement depth cues and easy search for revealing viewpoints similar to turning a physical object in the hand to examine it, in addition to the perspective and stereo aspects of the individual rendered frames. In certain implementations, the stereo depth cue may be omitted, leaving the user to rely on these other depth cues. These may be useful for a one-eyed user, or a user whose brain does not process stereo cues effectively.

As will be appreciated by those skilled in the art, every image acquisition system has certain constraints. Aspects of the present technique advantageously embed these limitations into the associated software so that the image selection process works with the specific imaging modality. For example, while an MR scanner can acquire a slice image at an arbitrary angle, the limits on its spatial resolution make it difficult to specify an extremely small field of view (FOV) or volume. The aspects of the present technique embed this constraint in the software, limiting zoom and the size to which a selection may be reduced. Similarly, systems such as phased array radar or ultrasound, acquire data in a fan or cone shape (typically with circular or rectangular cross-section) with apex at the emission component. For such modalities, the user selecting a planar view may rotate the selection ‘fan’ among its possible positions, and use widgets such as corner-grabbing to widen or narrow it and to move the far and near spatial limits on the points for which data are to be collected. A CT system may not be able to acquire oblique planes, but the region over which it gathers planar or volume data has a geometry which by the present technique the user may select directly, modifying it by global translation and by dragging widgets that specify its boundaries, without being permitted to specify an unrealizable shape.

In some imaging modalities a wider range of selections may be possible by robotically mechanical motion of the system or its parts, such as the table on which a patient lies in a CT or MR scanner, rather than by electronic switching alone. In one exemplary embodiment the user may specify either a ‘switch mode’, and work with the changes that can be realized though electronic control, or a ‘mechanical mode’ in which there must be physical motion of the imaging system or its part. In mechanical mode, the user, according to aspects of the present technique may be able to select quickly a configuration to which the imaging system needs to move (implicitly, since the user specifies the results and the system computes how to get them) that is less likely than in a traditional system to require a new choice, and the time cost of new movement or the quality cost of accepting a sub-optimal scan.

Further, aspects of present technique may include other features in the virtual user interface, such as but not limited to head-tracking, or ‘haptic’ feedback, which uses the hand-held device (stylus) to deliver a force by which the user feels that the corresponding image is interacting with other elements of the scene (by striking, pulling, cutting, and the like). A microphone may be included, with devices and software internal to the processor by which sound may be recorded or analyzed. Multiple position-reporting devices may be used, and these may be attached to separate parts of the hand so that the current shape of the hand (closed, open, grasping between finger and thumb, and the like) may be reconstructed, and a hand in the corresponding position may included in the display.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An imaging system comprising:
a data acquisition system for obtaining a three-dimensional image of an object; and
a processor coupled to the data acquisition system, the processor configured for receiving a user interface input based on interaction with the three-dimensional image, and for providing a plurality of parameters to the data acquisition system based on the user interface input, the plurality of parameters being used for further acquisition by the data acquisition system.

2. The imaging system of claim 1 wherein the imaging system is at least one of a magnetic resonance system, a computed tomography system, an ultrasound system, an X-ray system, a magnetic resonance spectroscopy system, a radar system, a seismological system, an optical system, a microscope or a combination thereof.

3. The imaging system of claim 1 wherein the imaging system is used for at least one of industrial or medical applications, and wherein the medical applications comprise at least one of cardiac imaging applications, surgical planning applications, internal organ segmentation applications, anatomical connectivity applications, and directed connectivity applications.

4. The imaging system of claim 1 wherein the user interface input is obtained via a virtual user interface.

5. The imaging system of claim 4 wherein the virtual user interface comprises:
a computer workstation configured for displaying the three-dimensional image of the object;
a three-dimensional tracking device coupled to the computer workstation and configured for allowing up to six degrees of freedom of movement in a user interface input; and
a virtual display setup coupled to the computer workstation and configured to allow a user to reach in and interact with the three-dimensional image of the object via the three-dimensional tracking device.

6. The imaging system of claim 5 wherein the virtual user interface comprises a plurality of user options for selecting the user interface input, and wherein the user options comprise at least one of a slice, a triplane, a heart model, a zoom slider or a combination thereof.

7. The imaging system of claim 1 wherein the processor is further configured for image analysis, the image analysis comprising at least one of visualization, segmentation, fusion, or registration of the three-dimensional image of the object.

8. A virtual user interface comprising:
a computer workstation configured for displaying a three-dimensional image of an object;
a three-dimensional tracking device coupled to the computer workstation and configured to allow six degrees of freedom of movement in a user interface input;
a virtual display setup coupled to the computer workstation and configured to allow a user to reach in and interact with the three-dimensional image of the object via the three-dimensional tracking device; and
a processor adapted to be coupled with an imaging system and the computer workstation, the processor being configured to receive user interface input based on interaction with the three-dimensional image, and to provide a plurality of parameters to the data acquisition system based on the user interface input, the plurality of parameters being used for further acquisition by the data acquisition system.

9. The virtual user interface of claim 8 wherein the virtual display setup comprises a stereo display for providing distinct views to each eye of the user.

10. The virtual user interface of claim 8 further comprising a haptic device configured for providing feedback in a form of force felt by the user while interacting with the three-dimensional image of the object.

11. The virtual user interface of claim 8 further comprising a head-tracker for using position of head of the user to reach in and interact with the three-dimensional image.

12. The virtual user interface of claim 8 further comprising a microphone configured for at least one of recording sound from the object or for giving oral instructions for interacting with the three-dimensional image.

13. The virtual user interface of claim 8 further comprising a mirror oriented at a selected angle and configured for allowing the user to move the three-dimensional tracking device in the virtual display set-up without masking the display from the user.

14. An MR imaging system comprising:
an array of radio frequency coils for producing controlled gradient field and for applying excitation signals to a region of interest in a patient;
at least one detecting coil for detecting magnetic resonance signals resulting from the excitation signals;
a control circuit configured to energize the array of radio frequency coil;
a data acquisition system for obtaining a three-dimensional representation of the region of interest from the magnetic resonance signals detected by the at least one detecting coil; and
a virtual user interface comprising:
a computer workstation configured for displaying a three-dimensional representation of an object,
a three-dimensional tracking device coupled to the computer workstation and configured for allowing up to six degrees of freedom of movement in a user interface input,
a virtual display set-up coupled to the computer workstation and configured for allowing a user to reach-in and interact with the three-dimensional representation of the object via the three-dimensional tracking device, and
a processor adapted to be coupled with an imaging system and the computer workstation, the processor being configured to receive the user interface input based on interaction with the three-dimensional image, and to provide a plurality of parameters to the data acquisition system based on the user interface input, the plurality of parameters being used for further acquisition by the data acquisition system.

15. The MR imaging system of claim 14 wherein the plurality of parameters include at least three parameters from the x, y and z coordinates of a centre point of a location, and the roll, pitch and yaw of an orientation selected by the user via the three-dimensional tracking device.

16. The MR imaging system of claim 14 wherein the processor is further configured for creating a movie to view continually successive three-dimensional images of a plurality of regions of interest.

17. The MR imaging system of claim 14 further comprising image analysis, the image analysis comprising at least one of visualization, segmentation, fusion, or registration of at least one three-dimensional representation of a region of interest.

18. The MR imaging system of claim 14 wherein the virtual user interface further comprises a mirror oriented at a selected angle and configured for allowing the user to move the three-dimensional tracking device in the virtual display set-up without masking the display from the user.

19. A method of acquiring three-dimensional data in an imaging modality, the method comprising:
obtaining a three-dimensional image of an object being imaged;
receiving a user interface input based on interaction with the three-dimensional image; and
providing a plurality of parameters to a data acquisition system based on the user interface input.

20. The method of claim 19 further comprising providing a plurality of user options for interacting with the three-dimensional image, and wherein the user options comprise at least one of a slice, a triplane, a heart model, a zoom slider or a combination thereof.

21. The method of claim 19 further comprising analyzing an image wherein analyzing comprises at least one of visualization, segmentation, fusion, or registration of the three-dimensional image of the object.
